(19) United States
(12) Patent Application Publication
(10) Pub. No.: US 2012/0202863 A1
(43) Pub. Date: Aug. 9, 2012

(54) Compositions and methods for treatment of glaucoma

(76) Inventor: Gerald Horn, Deerfield, IL (US)

(21) Appl. No.: 12/931,632

(22) Filed: Feb. 3, 2011

(51) Int. Cl. A61K 31/4164; A61P 27/02; A61P 27/06

(52) U.S. Cl. 514/396

(57) ABSTRACT

The invention provides α-2 adrenergic receptor agonist compositions and methods for treating glaucoma and other intraocular conditions. The preferred α-2 agonist used in the inventive compositions and methods is dexmedetomidine at near alkaline pH and extremely low concentrations.

The file of this patent contains at least one drawing executed in color. Copies of this patent with color drawing(s) will be provided by the Patent and Trademark Office upon request and payment of the necessary fee.

### BACKGROUND OF THE INVENTION

Glaucoma is a multifactorial disease which encompasses a spectrum ranging from elevated intraocular pressure (IOP) to reduced vascular perfusion of the optic nerve.

While many factors have been implicated as contributing causes of glaucoma, currently existing treatments for glaucoma have limited effectiveness in lowering IOP and/or are accompanied by a number of side effects, such as fatigue, sedation, lid allergy, topical allergy, and/or redness.

Because of the side effects, an additional major problem in glaucoma therapy is patient compliance in taking medications as prescribed. It is believed that many of these side effects and suboptimal efficacy of the existing treatments are unintended consequences of alpha-1 (α-1) receptor induction from treatment with alpha agonists.

Accordingly, there is a need for novel formulations of alpha-2 (α-2) agonists for the treatment of glaucoma which would have minimal, if any, cross-activation of α-1 receptors, more effective IOP lowering with significantly reduced or eliminated side effects of conventional α-2 agonists, such as sedation and redness.

### SUMMARY OF THE PRESENT INVENTION

The present invention provides compositions and methods effective for the treatment of glaucoma in a patient in need thereof. Preferably, the compositions of the invention are formulated to prevent sedation, eliminate or reduce redness, eliminate or reduce ocular allergy, as well as significantly reduce intraocular pressure.

In some embodiments, the provided compositions may also have an eye whitening effect. Most preferably, the compositions include all of the above benefits and also have neuroprotective benefits and may be used for optic nerve protection, including the treatment of neurodegenerative conditions, such as ischemic optic neuropathy, diabetic retinopathy, optic ischemia, and other optic neuropathies, particularly those involving retinal ganglion cells and/or axons at or near the optic nerve lamina.

The present invention optimizes α-2 agonist corneal permeation utilizing a highly selective α-2 agonist which is formulated to have a high topical and intraocular lipophilicity of preferably 2.5 or greater.

The preferred compositions of the invention employ selective α-2 adrenergic receptor agonists which share some or all of the following characteristics:
a) a high selectivity for α-2 over α-1 adrenergic receptors, such as 1000:1 or greater; more preferably 1500:1 or greater; and even more preferably 2000:1 or greater;
b) a very low concentration, such as from between about 0.001% to about 0.025%; more preferably, between about 0.0025% to about 0.025% weight by volume; more preferably between about 0.0035% and about 0.018% weight by volume, and still more preferably about 0.0065% to about 0.0125% weight by volume;
d) alkalinity, such as an alkaline or near alkaline pH of higher than 6.5; preferably between about 6.5 to 7.5; and more preferably between about 6.7 and about 7.0;
e) a high degree of intraocular lipophilicity as measured by the Log P, an octanol-water partition coefficient, such as Log P of between about 2.40 and 3.00; and more preferably between about 2.50 and 2.85 at physiologic pH;
f) a higher degree of corneal penetration than that of conventional α-2 adrenergic receptor agonists-based glaucoma drugs as measured, for example, by the Log D, a measure of topical lipophilicity based on the octanol-water partition coefficient vs. pH, being between 2.40 and 3.0, and more preferably between 2.50 and about 2.85; and
g) a substantially greater solubility in an aqueous solution which allows to achieve the required concentration range of 0.002% (0.02 mg/cc) to 0.02% (0.2 mg/cc); and allows for improved solubility and stability over a range of temperatures, where such solubility is preferably about 1 mg/cc or greater at the preferred pH range of 6.7 or greater.

Preferably, the compositions of the invention contain corneal penetration enhancers. Corneal penetration agents include, but are not limited to, a citrate salt and/or other salts which increase solubility, chelating agents, preservatives, ion-channeling agents, cyclodextrin, or other additives which increase corneal permeability.

It is currently believed that the most preferred selective α-2 adrenergic receptor agonist suitable for purposes of the invention is dexmedetomidine in specific formulations which meet the above-listed characteristics. Accordingly, in some embodiments, compositions and methods of the invention include dexmedetomidine, or another selective α-2 adrenergic receptor agonist, at a concentration from between about 0.0025% to about 0.025% weight by volume.

In some embodiments, dexmedetomidine, or another selective α-2 adrenergic receptor agonist, has an octanol-water partition coefficient of between about 2.40 and 3.00; and more preferably, between about 2.50 and 2.85.

In the most preferred embodiment, the invention provides a pharmaceutical composition for the treatment of glaucoma which includes:
a. dexmedetomidine at a concentration from between about 0.001% to about 0.025% weight by volume; and any two of the following:
b. a corneal penetration/solubility enhancer, such as a salt selected from the group consisting of citrate, mesylate, hydrobromide/bromide, acetate, fumarate, sulfate/bisulfate, succinate, phosphate, maleate, nitrate, tartrate, benzoate, carbonate, and pamoate; preferably at a concentration of between about 0.1% and 0.5%, and more preferably between about 0.15% to 0.20%; and/or
c. carboxymethyl cellulose at a concentration of between about 0.05% and about 0.5% weight by volume, most preferably at 0.1%; and/or
d. mannitol at a concentration of between about 1% and about 10% weight by volume, most preferably at 4% and/or
e. 2-hydroxypropyl-beta cyclodextrin at a concentration of between about 0.5% and about 5% weight by volume; and/or
f. Tween® 80 detergent (or other Tween® detergent, including polyethylene glycol, propylene glycol, polyvinyl alcohol and glycerin; and/or
g. preservatives, including solubility enhancers, such as methylparaben, propylparaben, benzalkonium chloride (BAK) and ethylenediaminetetraacetic acid (EDTA), preferably at a concentration of between 0.01% and 0.05%, most preferably 0.02%; and
h. buffers, wherein said pharmaceutical composition has a pH of about 6.5-7.5, and more preferably about 6.7; and where alkaline or near alkaline buffers may be used, such as acetate, citrate, phosphate, borate, carbonate, or others.

The composition may further include other stabilizing agents and/or other additives as more fully described below.

In some embodiments, compositions and methods of the invention include selective α-2 adrenergic receptor agonists which have binding affinities (Kᵢ) for α-2 over α-1 receptors of 1000 fold or greater and have an octanol-water partition coefficient of about 2.50 or greater.

In yet other embodiments, compositions and methods of the invention include selective α-2 adrenergic receptor agonists which have Kᵢ for α-2 over α-1 receptors of 1000 fold or greater and are at a concentration from between about 0.001% to about 0.025% weight by volume.

In some embodiments, compositions and methods of the invention include selective α-2 adrenergic receptor agonists which have Kᵢ for α-2 over α-1 receptors of 1500 fold or greater, are present at a concentration from between about 0.001% to about 0.025% weight by volume, and have pH of about 7.0 or greater.

In some embodiments, the compositions of the invention may also include other therapeutic agents; however, the compositions are intended to be effective without the need for any other therapeutic agents, specifically including, but not limited to, α-1 antagonists.

The invention also provides methods of treating and/or preventing glaucoma with the provided compositions. The provided methods lower IOP in glaucoma patients, reduce redness, and provide eye whitening.

### BRIEF DESCRIPTION OF THE FIGURES

FIG. 1A is a photograph of a patient's eyes following application of 0.01% dexmedetomidine;

FIG. 1B is a photograph of a patient's eyes immediately prior to application of 0.0065% dexmedetomidine;

FIG. 1C is a photograph of a patient's eyes immediately after application of 0.0065% dexmedetomidine;

FIG. 2A is a photograph of a patient's eyes immediately prior to application of 0.0133% dexmedetomidine (OS; left eye) and Xalatan® (latanoprost) (OD; right eye);

FIG. 2B is a photograph of the same eyes as in FIG. 2A 5 minutes after application of 0.0133% dexmedetomidine (OS; left eye) and Xalatan® (latanoprost) (OD; right eye); and

FIG. 2C is a photograph of the same patient's eyes as in FIG. 2A following 3 hour application of 0.0133% dexmedetomidine (OS; left eye) and Xalatan® (latanoprost) (OD; right eye).

### DETAILED DESCRIPTION OF THE INVENTION

### Definitions

The term “α-1 adrenergic receptor” refers to a G-protein-coupled receptor (GPCR) associated with the G_{q} heterotrimeric G-protein.

The term “α-2 adrenergic receptor” refers to a GPCR associated with the Gᵢ heterotrimeric G-protein.

The term “selective α-2 adrenergic receptor agonists” encompasses all α-2 adrenergic receptor agonists which have a binding affinity of 1000 fold or greater for α-2 over α-1 adrenergic receptors, and more preferably 1500 fold or greater. The term also encompasses pharmaceutically acceptable salts, esters, prodrugs, and other derivatives of selective α-2 adrenergic receptor agonists.

The term “dexmedetomidine” encompasses, without limitation, dexmedetomidine salts, esters, prodrugs and other derivatives.

The term “prodrug” refers to a compound that may be converted under physiological conditions to a biologically active compound.

As used herein, the term “composition” is intended to encompass a product comprising the specified ingredients in the specified amounts, as well as any product which results, directly or indirectly, from a combination of the specified ingredients in the specified amounts.

The terms “treating” and “treatment” refer to reversing, alleviating, inhibiting, or slowing the progress of the disease, disorder, or condition to which such terms apply, or one or more symptoms of such disease, disorder, or condition.

The terms “preventing” and “prevention” refer to prophylactic use to reduce the likelihood of a disease, disorder, or condition to which such term applies, or one or more symptoms of such disease, disorder, or condition. It is not necessary to achieve a 100% likelihood of prevention; it is sufficient to achieve at least a partial effect of reducing the risk of acquiring such disease, disorder, or condition.

The term “significant side effects” refers to substantial side effects of the treatment which include at least: a) sedation of a patient such that the patient feels sedated and becomes impaired or b) visually noticeable increase in redness of a patient's eye due to hyperemia.

The term “medicamentosa” refers to the inflammatory sequelae of α-1 agonist topical medications, particularly following topical ocular or nasal delivery, such as the development of increased vasodilation and hyperemia, requiring more frequent instillation of topical vasoconstrictor, resulting in a cyclically increasing ischemia and, eventually, a persistent adverse toxicity lasting weeks to months even after drug discontinuation.

### Embodiments of the Invention

The present invention provides compositions and methods effective for the treatment of glaucoma in a patient in need thereof. Preferably, the compositions of the invention are formulated to prevent sedation, eliminate or reduce redness, as well as more significantly reduce intraocular pressure than prior art formulations of α-2 agonists.

The compositions of the invention provide considerable IOP lowering improvement over prior art α-2 agonists, particularly for the eyes of glaucoma patients, may increase duration of therapeutic action and reduce the incidence of rebound hyperemia and/or other allergic reaction. They also further improve cosmetic appearance (for example, increasing whiteness and providing additional whitening) of the treated eyes, resulting in improved patients' compliance; and provide optic nerve protection, retinal ganglion cell neuroprotection, an increase in α-2 agonist concentration in the inner retinal plexiform, and additional neuroprotective benefits. They may also increase the outflow at the trabecular meshwork which is populated with endothelial cells and believed to be populated with α-2a receptor in humans.

Further advantages of the inventive compositions and methods include reduction of topical and systemic side effects, including but not limited to oral dryness, ocular hyperemia, burning and stinging, headache, blurring, foreign body sensation, fatigue/drowsiness, conjunctival follicles, ocular allergic reactions, ocular pruritus, corneal staining/erosion, photophobia, eyelid erythema, ocular ache/pain, ocular dryness, tearing, upper respiratory symptoms, eyelid edema, conjunctival edema, dizziness, blepharitis, ocular irritation, gastrointestinal symptoms, asthenia, conjunctival blanching, abnormal vision, muscular pain, lid crusting, conjunctival hemorrhage, abnormal taste, insomnia, conjunctival discharge, depression, hypertension, anxiety, palpitations/arrhythmias, nasal dryness and syncope.

In some embodiments, the compositions and methods of the invention significantly lower intraocular pressure while at the same time reducing redness and providing eye whitening. Some of the observed intraocular pressure lowering effects include:
1) onset within one hour;
2) peak effects of 40%-44% reduction over low-normal baseline IOP of 13-18;
3) peak effects at hour 2-hour 5;
4) prolonged action with over 20% reduction over baseline at hour 8; and over 15% reduction at 26 hours from a single dose; and
5) improved cosmetic appearance via reduction of redness and in some cases cosmetic whitening.

It is believed that the inventive formulations provide a combination of a very high α-2 selectivity and very low concentration to enhance glaucoma therapy by reducing and/or eliminating unintended stimulation of intraocular α-1 receptors.

In order for a subclass of α-2 agonists to have superior and previously unknown ocular hypotensive (i.e., reducing IOP) and other therapeutic benefits for the treatment of glaucoma (including all forms of open angle glaucoma, ocular hypertension, pseudoexfoliative glaucoma, and neovascular glaucoma), a specific optimized combination of high α2/α1 selectivity, a high degree of corneal penetration, optimized topical lipophilicity, intraocular lipophilicity, significant minimum topical alkalinity and an extreme low dose is preferred. While a drug that satisfies any of these characteristics may work, there is an enhanced amplified benefit achieved when all of these characteristics are met.

The present invention provides both: 1) enhanced intraocular α-2 receptor agonist effects; and 2) reduction or elimination of unintended adverse induction of α-1 receptors. It is believed that the reduction or elimination of significant side effects is possible because the inventive compositions do not activate α-1 receptors, increase intraocular penetration, and increase intraocular binding affinity to cell membranes while decreasing the topical concentration required. Because the present invention maximizes the potential of α-2 agonists, it provides compositions and methods to treat glaucoma which do not require a second therapeutic agent, such as an α-1 antagonist.

Achieving intraocular α-2 effects without inducing α-1 topical or intraocular ischemic effect (i.e., restriction of blood supply) promotes the full spectrum of α-2 agonist activity benefits for treating glaucoma including the following: 1) reduced level of pro-inflammatory cytokines; 2) reduced direct general α-1 induced ischemia to retinal ganglion cells and optic nerve fibers, to which the optic nerve, particularly along the lamina cribosa, may be extremely sensitive; 3) selective microvessel constriction and aqueous synthesis reduction without α-1 induced vasoconstriction and attendant ischemia; 4) greater intraocular accumulation, diffusion, and concentrated delivery of more α-2 and less α-1 selective drug in lipophilic ciliary body-iris pigment epithelium and retinal pigment epithelium where it is most needed for the present invention; and 5) greater permeation and/or duration of action on intraocular α-2 receptors by helping a drug to more readily penetrate cell membranes.

The preferred selective α-2 adrenergic receptor agonists share some or all of the following characteristics:
a) a high selectivity for α-2 over α-1 adrenergic receptors, such as 1000:1 or greater; more preferably 1500:1 or greater; and even more preferably 2000:1 or greater;
b) a very low concentration, such as from between about 0.001% to about 0.025%; more preferably, from between about 0.0025% to about 0.025% weight by volume; more preferably between about 0.0035% and about 0.018% weight by volume, and still more preferably about 0.0065% to about 0.013% weight by volume;
d) alkalinity, such as an alkaline or near alkaline pH of between about 6.5 to 7.5; and more preferably between about 6.7 and about 7.0;
e) a high degree of intraocular lipophilicity as measured by the Log P, an octanol-water partition coefficient, such as Log P of between about 2.40 and 3.00; and more preferably between about 2.50 and 2.85 at physiologic pH;
f) a higher degree of corneal penetration than that of conventional α-2 adrenergic receptor agonists-based glaucoma drugs as measured, for example, by the Log D, a measure of topical lipophilicity based on the octanol-water partition coefficient vs. pH, being between 2.40 and 3.0, and more preferably between 2.50 and about 2.85; and
g) a substantially greater solubility in an aqueous solution which allows to achieve the required concentration range of 0.002% (0.02 mg/cc) to 0.02% (0.2 mg/cc); and allows for improved solubility and stability over a range of temperatures, where such solubility is preferably about 1 mg/cc or greater at the preferred pH range of 6.7 or greater.

Administration of α-2 agonists with a high octanol-water partition coefficient at a too high concentration, (i.e., substantially higher than 0.025%) can lead to α-1 induced adverse effects, including pressure spikes, ischemia, adverse cytokine increase, adverse neuronal degenerative effects, sedation and other undesired side effects, such as redness, hyperemia, systemic hypotension, bradycardia, etc.

Preferably, the compositions of the invention employ selective α-2 agonists at a very low concentration, such as from between about 0.001% to about 0.025% weight by volume; and more preferably between about 0.0065% and about 0.018% weight by volume.

It is currently believed that the most preferred selective α-2 adrenergic receptor agonist suitable for purposes of the invention is dexmedetomidine as either the HCL salt (preferably, with citric acid or its salt as an additive), or as the citrate salt. Other salts may similarly be substituted for the HCl.

Accordingly, in some embodiments, compositions and methods of the invention include dexmedetomidine, or another selective α-2 adrenergic receptor agonist, at a concentration from between about 0.001% to about 0.025% weight by volume.

### Selectivity for α-2 Versus α-1 Adrenergic Receptors

The selective α-2 adrenergic receptor agonists have binding affinities (Kᵢ) for α-2 over α-1 receptors of 1000:1 or greater; more preferably 1500:1 or greater; and even more preferably 2000:1 or greater. It is well within a skill in the art to design an assay to determine α-2/α-1 functional selectivity. For example, potency, activity or EC₅₀ at an α-2A receptor can be determined by assaying for inhibition of adenylate cyclase activity. Furthermore, inhibition of adenylate cyclase activity can be assayed, without limitation, in PC12 cells stably expressing an α-2A receptor such as a human α-2A receptor. Additionally, potency, activity or EC₅₀ at an α-1A receptor can be determined by assaying for intracellular calcium. Intracellular calcium can be assayed, without limitation, in HEK293 cells stably expressing an α-1A receptor, such as a bovine α-1A receptor.

For the purposes of the present invention, it is desired to avoid or minimize triggering of α-1 receptors. Even a small critical threshold achieved of undesired α-1 receptor recruitment creates sufficient generalized vasoconstriction, micro-inflammatory change, and/or pro-inflammatory cytokine release to reduce effectiveness of the α-2 receptor induced positive treatment effects.

It is a discovery of the present invention that at very low concentrations highly lipophilic selective α-2 agonists still have a sufficiently strong activation of α-2 receptors with minimal or no cross-activation of α-1 receptors. Accordingly, when these α-2 agonists are used for the treatment of glaucoma, they greatly reduce IOP and provide eye whitening without significant side effects believed to be associated with α-1 receptors, such as rebound hyperemia.

In some embodiments, compositions and methods of the invention include selective α-2 adrenergic receptor agonists which have Kᵢ for α-2 over α-1 receptors of 1500 fold or greater and have an octanol-water partition coefficient of about 2.50-3.0 both at topical pH (Log D) and intraocular pH (Log P).

In yet other embodiments, compositions and methods of the invention include selective α-2 adrenergic receptor agonists which have Kᵢ for α-2 over α-1 receptors of 1000 fold or greater and are at a concentration from between about 0.0025% to about 0.025% weight by volume.

It is further preferred that α-2 agonists preferably target α-2a receptors as compared to α-2b or α-2c receptors.

Brimonidine, guanfacine, guanabenz, dexmedetomidine and fadolmidine are some of the sufficiently highly selective α-2 agonists to satisfy the selectivity requirement.

### Lipophilicity

Lipophilicity may be measured, for example, using known measurements, such as log P (log K_{OW}) derivation of the octanol-water partition coefficient and/or, a closely related coefficient, X Log P3-AA. See, for example, Tiejun Cheng et al, Computation of Octanol-Water Partition Coefficients by Guiding an Additive Model with Knowledge, J. Chem. Inf. Model., 2007, 47 (6), pp 2140-2148. These measurements represent the intraocular lipophilicity value of topical drugs for intraocular delivery (i.e., once the drug permeates into the anterior chamber). A person of ordinary skill in the art is well familiar with these measurements.

It is believed that lipophilicity of an α-2 agonist compound is related to pH: the more alkaline pH, the more lipophilic the compound particularly, for α-2 agonists with pK values of near or greater than 7.0. This is because at more alkaline pH, more of the compound is present in a non-ionized form. Usually, Log P and/or X Log P3-AA are measured when the formulation at issue is or will be at the physiologic pH of about 7.4.

The preferred Log P (and X Log P3-AA values) suitable for the purposes of the invention are between about 2.50 and 5.00; and more preferably, between about 2.75 and 3.50. At this range, an α-2 agonist is highly lipophilic and may more easily penetrate lipophilic cell membranes where α-2 receptors are found. Further, its binding affinity to such receptors is increased and, because of depot diffusion from lipophilic intraocular structures (such as ciliary body, iris pigment and retinal pigment epithelium), its efficacy is enhanced.

If the selectivity of a specific α-2 agonist is substantially above 1600:1 (for example, 2000:1), then it is possible that this agonist may be effective for the purposes of the invention even if it has a slightly lower octanol-water partition coefficient.

In some embodiments, dexmedetomidine, or another selective α-2 adrenergic receptor agonist, has Log P at pH 7.4 of about 2.50 or greater; preferably, between about 2.75 and 5.00; and more preferably between about 2.75 and 3.50 for intraocular lipophilicity.

Log D refers to a lipophilicity value at a given pH. This measurement is especially useful to determine the level of topical lipophilicity and resultant corneal permeability of a topical composition. Corneal permeability is a complex event, which may be affected by polar surface area, H⁺ donor activity, bond rotation, and active transport phenomenon. It is a discovery of the present invention that the Log D values of between about 2.50 and about 3.00, and more particularly between about 2.66 and about 2.88 are preferred for increased corneal permeation of α-2 agonists.

In some embodiments, dexmedetomidine, or another selective α-2 adrenergic receptor agonist, has Log D of between about 2.50 and 3.00, and more preferably, between about 2.6 and about 2.9 at pH 6.5-7.1.

α-2 agonists are generally basic molecules with pKa values typically at or above 7.0. The increased Log D value for dexmedetomidine at pH above 6.4 enhances corneal penetration, and the Log P value of about 3.1 for this preferred embodiment substantially enhances its efficacy for glaucoma treatment.

A discovery of the present invention is that efficacy is so greatly enhanced at optimized Log D (topical lipophilicity) and Log P (intraocular lipophilicity) that concentrations well below those previously found effective can become effective.

Ideally, both Log P value (i.e., a measure of intraocular efficacy) and Log D value (a measure of corneal permeation) are optimized.

Table 1 provides known X Log P3-AA values and α2/α1 binding affinities for several α-2 agonists.

TABLE 1

| α-2 Agonist | XLogP3AA | α2:α1 |
|---|---|---|
| Brimonidine (0.15% pH 6.6-7.4; 0.10% | 0.6-1.8 | 976 |
| pH 7.4-8.0) | | |
| Guanfacine | 2.0 | |
| Guanabenz | 1.7 | |
| Dexmedetomidine | 3.1 | 1620 |
| Fadolmidine pivalyl prodrug ester | 1.8 | |
| Fadolmidine | 1.2 | |
| Methoxamine | 0.5 | |
| Oxymetazoline | 2.9 | 50 |
| Epinephrine | −1.4 | |
| Clonidine | 1.6 | 200 |
| Apraclondine | 1.3 | 150 |
| Mivazerol | 1.1 | |
| Xylazine | 2.8 | 160 |
| Methyl Dopa | −1.9 | |
| Lofexidine | 2.6 | <300 |

Table 1 demonstrates that among the listed α-2 agonists, only dexmedetomidine has an acceptable combination of X Log P3-AA and α2:α1 coefficient. However, it is possible that there may be other formulations including other α-2 agonists which meet the defined requirements of the present invention in both selectivity and lipophilicity categories.

The relationship between the α2/α1 selectivity and the lipophilicity means that for increasingly selective drugs a greater degree of suboptimal topical lipophilicity can still be efficacious. This relationship can be characterized by the following inventive equation:

The minimum acceptable Log P(KOW)=(3−0.5[α2:α1 ratio−1000]/1000)+/−0.2.

This equation provides the following values vs. α-2 selectivity:

Brimonidine (or analogue) (Kᵢ=1000 α2/α1): log P (log K_{OW})=3.00+/−0.2;

### Dexmedetomidine (Kᵢ=1600 α2/α1): log P (log K_{OW})=2.96+/−0.2; and

an α-2 agonist with Kᵢ=2000 α2/α1: log P (log K_{OW})=2.5+/−0.2

### Alkalinity

It is preferred that the compositions of the present invention be at a near alkaline or alkaline pH of 6.5 or greater; preferably between about 6.5 and about 7.5; preferably between about 6.6 and about 7.3; and more preferably between about 6.7 and about 6.9. In one embodiment, the increase from pH 6.4 to pH 7.0 or greater and preferably to 6.7 to 7.0 was discovered to potentiate glaucoma hypotensive effects of dexmedetomidine formulations, presumably due to increased drug corneal penetration.

A pH of a formulation of the invention is the final pH of the formulation. It is to be distinguished from the solution used as diluent, which may have a higher pH than the final pH of the formulation solubilized within the diluent. For example, for dexmedetomidine HCl, balanced salt solution BSS® (NaCl 0.64%, KCl 0.075%, CaCl₂*H₂O 0.048%, MgCl₂*6H₂O 0.03%, C₂H₃NaO₂*3H₂O 0.39%, C₆H₅Na₃O₇*2H₂O, Alcon Laboratories) has a pH of about 7.1, but produces a final pH of about 6.7, most likely due to increased ionized right shift with H⁺ release created by the sodium citrate dihydrate.

Normally, α-2 agonists are formulated as salts selected to improve solubility at acidic pH, and typically achieve an acidic pH when dissolved in water (pH of about 3.5-6.0). It is a surprising discovery of the present invention that a pH of 6.5 or greater is preferred for topical glaucoma therapy because of the exponential decrease in solubility in this range to about 0.022% or less, below known effective concentrations of α-2 agonist glaucoma drugs.

However, the alkaline pH also approximates or exceeds the pKa of dexmedetomidine at 7.1, thereby increasing the percentage of drug in non-ionized lipophilic form, as the Log D value increases and potentiates corneal penetration. When dexmedetomidine at the inventive formulations is administered, the heightened lipophilicity and α-2 selectivity of dexmedetomidine (a preferred α-2 agonist) at intraocular pH levels of about 7.4 (Log P value) increases intraocular peak hypotensive effect as well as duration of the effect versus other α-2 agonists.

Generally, one can vary the level of alkalinity of the compositions of the invention to achieve a preferred degree of topical lipophilicity, desired corneal permeation range, and desired intraocular lipophilicity.

For example, at a final pH of between 5.0 and about 6.4, a sufficiently high octanol-water partition coefficient for drug optimization for the present invention cannot be achieved with existing α-2 agonists. However, at a pH of between 6.5 and 7.5, selective α-2 agonists, e.g., dexmedetomidine, have an increasing Log P value which is preferred for the purposes of the present invention.

At a diluent pH of 7.1, the lipophilicity of dexmedetomidine is significantly increased to a Log P of 2.66.

### Corneal Permeation

The compositions of the present invention have an improved α-2 intraocular efficacy and corneal permeation. As a rough rule of thumb, if a drug's efficacy increases 50% but the drug's permeation decreases 50%, the net effect remains unchanged. Without wishing to be bound to a specific theory, it is believed that the improved efficacy and corneal permeation of the compositions of the present invention is largely due to their enhanced intraocular lipophilicity and optimized topical lipophilicity.

Certain additives, including weak acids, chelating agents, and cyclodextrins, can increase the corneal permeation. Once the additive components are disassociated from the active agent intraocularly (e.g., dexmedetomidine), the active agent of the present invention has intraocular efficacy associated with two variables: 1) proportional to the degree of α2:α1 selectivity and 2) proportional to the degree of intraocular lipophilicity (Log P).

The preferred combination of α2:α1 selectivity and Log P is believed to increase the α-2 agonist's membrane permeation to and within ciliary processes and trabecular meshwork. The preferred combination facilitates the α-2 agonist's target interaction, increases its binding affinity with the α-2 receptors and results in more durable therapeutic effects.

### Solubility

The solubility of α-2 agonists decreases exponentially at an increased pH. Table 2 illustrates the relationship between pH and solubility in water for dexmedetomidine. It shows that the soluble concentration of dexmedetomidine falls exponentially with higher pH.

TABLE 2

| pH | solubility (mg/ml) | max soluble concentration | BSS solution* |
|---|---|---|---|
| 6.0 | 1.953 | 0.195% | |
| 6.4 | ~0.60 | 0.060% | |
| 7.0 | 0.224 | 0.023% | ≧0.10% |
| 7.4 | ~0.150 | 0.015% | |
| 8.0 | 0.134 | 0.013% | |

*BSS = Balanced Salt Solution

In some embodiments of the present invention, it may be necessary to improve (i.e., increase) the solubility of α-2 agonists. A greater solubility has a number of advantages, including but not limited to an ability to achieve higher concentrations and enhanced stability at storage at cold temperatures. Because the desired concentration of suitable α-2 agonists is very low, and the present invention provides formulations with much greater solubility, the desired concentrations are easily achieved even at an exponentially reduced known solubility in the desired near-alkaline to alkaline pH range.

It is a surprising discovery of the present invention that α-2 agonists, and more specifically, dexmedetomidine, are rendered more effective as well as more soluble by constituents of a balanced salt solution. The terms “salt” and “constituent of a balanced salt solution” are used interchangeably for the purposes of the present invention. They are a subset of agents that improve solubility of the inventive formulations.

Thus, in one embodiment of the present invention, dexmedetomidine is rendered soluble up to or beyond 0.1% at pH 7.1 by adding constituents of a balanced salt solution. In a preferred embodiment, these constituents include any combination of one or more of the following: sodium citrate dehydrate, sodium acetate, and calcium salt. In a more preferred embodiment, the concentration of sodium dehydrate is about 0.17%; the concentration of sodium acetate is about 0.39%; and the concentration of calcium salt is about 0.048%.

The most preferred agent that improves solubility is a citrate salt. Citrate salt acts as a preservative and a corneal penetration enhancer. It has been further discovered that to achieve the desired final pH of around 6.4 to 6.7 and Log D values of above 2.50 of dexmedetomidine formulations, one may add a citrate salt at pH of about 7.1, preferably using an acetate or other similar pH range buffer.

Other agents that improve solubility which may be used for the purposes of the present invention include, but are not limited to, methanesulfonate (mesylate), hydrobromide/bromide, acetate, fumarate, sulfate/bisulfate, succinate, citrate, phosphate, maleate, nitrate, tartrate, benzoate, carbonate, pamoate, borate, glycolate, pivylate, sodium citrate monohydrate, sodium citrate trihydrate, sodium carbonate, sodium EDTA, phosphoric acid, penatsodium pentetate, tetrasodium etidronate, tetrasodium pyrophosphate, diammonium ethylenediamine triacetate, hydroxyethyl-ethylenediamine triacetic acid, diethylenetriamine pentaacetic acid, nitriloacetic acid, and various other alkaline buffering salts, polyanionic (multiple negatively charged) compounds, such as methylcellulose and derivatives, particularly carboxymethyl cellulose (CMC); and/or addition of cyclodextrins and/or their derivatives, particularly (2-Hydroxypropyl)—beta-cyclodextrin; certain solvents such as Tween 20, Tween 80, polyvinyl alcohol, propylene glycol and analogues or derivatives thereof; certain osmotic agents, such as mannitol or sucrose, HPMC or analogues and/or derivatives thereof, or certain chelating agents.

It is well within a skill of a skilled in the art to determine the amounts and concentrations of the agents improving solubility.

### Compositions and Methods of the Present Invention

Compositions and methods of the inventions encompass all isomeric forms of the described α-2 adrenergic receptor agonists, their racemic mixtures, enol forms, solvated and unsolvated forms, analogs, prodrugs, derivatives, including but not limited to esters and ethers, and pharmaceutically acceptable salts, including acid addition salts. Examples of suitable acids for salt formation are hydrochloric, sulfuric, phosphoric, acetic, citric, oxalic, malonic, salicylic, malic, furmaric, succinic, ascorbic, maleic, methanesulfonic, tartaric, and other mineral carboxylic acids well known to those in the art. The salts may be prepared by contacting the free base form with a sufficient amount of the desired acid to produce a salt in the conventional manner. The free base forms may be regenerated by treating the salt with a suitable dilute aqueous base solution such as dilute aqueous hydroxide potassium carbonate, ammonia, and sodium bicarbonate. The free base forms differ from their respective salt forms somewhat in certain physical properties, such as solubility in polar solvents, but the acid salts are equivalent to their respective free base forms for purposes of the invention. (See, for example S. M. Berge, et al., “Pharmaceutical Salts,” J. Pharm. Sci., 66: 1-19 (1977) which is incorporated herein by reference).

As long as a particular isomer, salt, analog, prodrug or other derivative of a suitable selective α-2 adrenergic receptor agonist functions as a suitable selective α-2 agonist, it may be used for the purposes of the present invention.

When choosing a particular α-2 adrenergic receptor agonist, one may take into account various considerations including any possible side effects and other systemic reactions.

The compositions of the present invention are preferably formulated for a mammal, and more preferably, for a human. In one embodiment of the invention, the compositions are delivered as ophthalmic solutions into the eyes. The invention also contemplates topical compositions which include, but are not limited to, gels and creams. They may also include additional non-therapeutic components, which include, but are not limited to, preservatives, delivery vehicles, tonicity adjustors, buffers, pH adjustors, antioxidants, tenacity adjusting agents, viscosity adjusting agents, and water.

The compositions of the invention may include various inactive ingredients commonly used in formulating topical compositions and that may improve stability of the formulation. For example, the compositions of the invention may include alcohols and/or surface active agents, including but not limited to polyglycol ether, polyethylene glycol-nonphenol ether, polyethylene glycol sorbitan monolaurate, polyethylene glycol sorbitan monooleate, polyethylene glycol sorbitanmonooleate, polyethylene glycol sterarate, polyethylene glycol polypropylene glycol ether, polyvinyl alcohol, polyvinyl pyrrolidine in a total amount of 0.05% to 5% by mass of the composition.

In some embodiments, the compositions of the invention may include acids or monoglycerides of fatty acids having 8 to 12 carbon atoms, which when in 0.5-1.5 M, and preferably equimolar concentration to the alpha 2 agonist may improve corneal permeation via ion pair formation; or antioxidants such as ion-exchange/photooxidation stabilizing agents, including but not limited to citric acid, sorbic acid, boric acid, caprylic acid, glyceryl monocaprylate, glyceryl monocaproate, glycerol monolaurate, sodium metabisulfite.

In some embodiments, the compositions and methods of the present invention may include chelating agents that further improve stability, including but not limited to ethylenediaminetetraacetic acid (EDTA) and structurally related acids and even more preferably citric acid or its salt. In some embodiments, the chelating agents are present at a concentration of between 0.02% and 0.2% weight/vol.

Preservatives include, but are not limited to, benzalkonium chloride (BAK), methylparaben, polypropylparaben, chlorobutanol, thimerosal, phenylmercuric acetate, perborate, or phenylmercuric nitrate.

Delivery vehicles include, but are not limited to, polyvinyl alcohol, polyethyleneglycol (PEG) and its analogues, povidone, hydroxypropyl methyl cellulose, poloxamers, carboxymethyl cellulose (CMC), hydroxyethyl cellulose and purified water. It is also possible to use a physiological saline solution as a major vehicle.

Tonicity adjustors include, but are not limited to, a salt such as sodium chloride, potassium chloride, dextran, cyclodextrins, mannitol, dextrose, glycerin, or another pharmaceutically or ophthalmically acceptable tonicity adjustor. In some embodiments, the tonicity modifying agents are present at a concentration of between 0.5% and 5% weight by volume.

The compositions of the present invention may comprise corneal permeation enhancing agents which include, but are not limited to, preservatives, cyclodextrins, viscosity enhancing agents, and ion-channel enhancing agents. In some embodiments of the invention, a corneal permeation enhancing agent may be selected from the group consisting of BAK at 0.01% to 0.02% weight by volume, EDTA at 0.01% weight by volume, caprylic acid, citric acid, boric acid, sorbic acid and/or salts, derivatives, and analogues thereof, where citric acid or its salt is a preferred embodiment.

Many of the listed additives (for example, BAK, EDTA, etc) may serve more than one purpose: for example, they can serve as both preservatives and corneal permeation enhancing agents (e.g. BAK), or solubilizing, preservative, and corneal permeation enhancing agents (e.g. citrate).

In some embodiments, the compositions and methods of the present invention may include viscosity agents and/or agents increasing solubility and/or stability, including but not limited to polyvinylpyrrolidone, methylcellulose, hydroxypropylcellulose, CMC, CMC sodium salt, gelatin, cellulose glycolate, sorbitol, alpha-cyclodextrin and/or other cyclodextrin derivatives, niacinamide, etc. In some embodiments, these agents are present at a total amount of 0.05% to 5% by w/v.

In a preferred embodiment, the amount of CMC is between about 0.05% and about 5%, and more preferably, between about 0.1% and about 0.3% weight by volume.

In a preferred embodiment, the amount of cyclodextrin is between about 0.1% and about 20%, and more preferably, between about 0.2% and about 0.5% weight by volume. In another preferred embodiment, the preservatives concentrations are: BAK 0.02%, or BAK 0.01% and EDTA 0.01%, where all units are weight by volume. In another preferred embodiment, the caprylic acid concentration is equimolar to that of dexmedetomidine, adjusted to optimize pH at about 6.7-6.9.

The compositions of the invention may also comprise a solubility stabilizer which preferably contains an anionic component, such as CMC, HPMC, or peroxide class preservatives. The solubility stabilizer allows one to achieve greater penetration of lipophilic membranes. In a preferred embodiment, the solubility stabilizer comprises a stabilized oxychloro complex, chlorite, and sodium perborate.

Buffers and pH adjustors include, but are not limited to, acetate buffers, carbonate buffers, phosphate buffers and borate buffers. It is understood that various acids or bases can be used to adjust the pH of the composition as needed. pH adjusting agents include, but are not limited to, sodium hydroxide and hydrochloric acid.

In one embodiment, the concentration of a tonicity agent and/or electrolyte concentration is within 280-320 mOsm range.

Antioxidants include, but are not limited to, sodium metabisulfite, sodium thiosulfate, acetylcysteine, butylated hydroxyanisole and butylated hydroxytoluene.

To make the topical compositions of the present invention, one can simply dilute more concentrated solutions of selective α-2 agonists, using methods known in the art with diluent of a balanced salt solution consisting of one or more of the above electrolytes or tonicity enhancing agents and preferably one or more of the above weak acids and or their salts to achieve a formulated pH of 6.50-7.00, and more preferably 6.6-6.8. The precise method of carrying out the dilutions is not critical. Any commonly used diluents, including preservatives described above in the application, suitable for topical solutions can be used.

The preferred compositions of the invention may include the following components:
dexmedetomidine at 0.0025%-0.025%, most preferably 0.0065%:0.0085% weight by volume;
mannitol at 1%-5%; most preferably 4%;
CMC at 0.1%-0.5%, most preferably 0.15%;
propylene glycol or polyvinyl alcohol at 1%-5%, most preferably 1.5% preferred;
sodium citrate dehydrate 0.1-0.2%
sodium borate and/or boric acid as buffer;
caprylic acid, preferably equimolar to dexmedetomdine; and
EDTA at 0.005%-0.1%; preferably at 0.01%.

In some embodiments, the compositions of the inventions include sodium chloride at a concentration of between about 0.3% and about 0.75% and/or potassium chloride at a concentration of between about 0.05% and about 0.15% and/or calcium chloride at a concentration of between about 0.02% and about 0.05%

In some embodiments, the invention provides the following compositions:

1. dexmedetomidine 0.0075%-0.0133%
diluent: balanced salt solution or 0.9% saline solution
0.02% BAK
Sodium citrate dehydrate 0.1-0.2%
Diluent pH 7.1, final pH of about 6.7
Acetate, borate or carbonate buffer 1 mM-100 mM; 10 mM preferred

2. dexmedetomidine 0.0080%
diluent: balanced salt solution or 0.9% saline solution
0.01% BAK
0.01% EDTA
mannitol 4%
final pH 6.8

3. dexmedetomidine 0.0065%
diluent: balanced salt solution or 0.9% saline solution
CMC (carboxymethylcellulose) 0.15%
0.02% BAK
0.01% EDTA
Final pH 6.7

4. dexmedetomidine 0.0070%
propylene glycol 1.0%
mannitol (as needed to create 290 mOsm) about 1%
caprylic acid, or glyceryl monocaprylate, or monocaproate equimolar to dexmedetomidine
CMC: 0.15%
0.02% BAK
0.01% EDTA
diluent: saline 0.70% (7 mg/ml) NaCl (optionally) and any or all of:
KCl 0.05% (0.50 mg/cc), CaCl₂ 0.07% (0.075 mg/cc), MgCl₂ 0.0037% (0.037 mg/cc)
Final pH 6.6

5. dexmedetomidine 0.0070%
propylene glycol or polyvinyl alcohol, 1.5%
mannitol as osmotic agent (as needed to create 290 mOsm) about 1%
citric or boric acid 0.1%-0.2%
CMC: 0.15%
0.02% BAK
0.01% EDTA
diluent: saline 7 mg/cc (0.70%) NaCl optionally and any or all of:
KCl 0.05% (0.50 mg/cc), MgCl₂ 0.0037% (0.037 mg/cc)
pH 6.9

6. dexmedetomidine 0.0075%
diluent: balanced salt solution or 0.9% saline solution
0.02% BAK
0.01% EDTA
Sodium citrate dehydrate 0.2%
Caprylic acid equimolar to dexmedetomidine 0.0075%
Acetate buffer 1-100 mM, preferably about 10 mM
Adjusted pH to 6.7 final

In the most preferred embodiment, the compositions of the invention include
a. dexmedetomidine at a concentration from between about 0.002% to about 0.02% weight by volume;
b. acetate salt at a concentration of 0.25%-0.50%;
c. Sodium citrate dehydrate 0.1-0.2%;
d. BAK 0.02% or BAK 0.01% and EDTA 0.01%
wherein said pharmaceutical composition has a final pH of about 6.5-7.0 or preferably 6.6-6.9, typically requiring for the above formulation a diluent of about pH 7.1.

The following Examples are provided solely for illustrative purposes and are not meant to limit the invention in any way.

### Example 1

### Effect of Topical Administration of Dexmedetomidine at Estimated Concentration of 0.0055% and 0.0065% on IOP

### Experimental Design

The purpose of this experiment was to evaluate the effect of topical ocular delivery of dexmedetomidine at concentrations of 0.0055% and 0.0065% on IOP. The experiment was designed as follows:

A total of four patients were given one drop of dexmedetomidine at pH 7.4 at either 0.0055% or 0.0065% concentration in one or both eyes at various time points. One of the four patients was administered both concentrations of dexmedetomidine (on different days), while the other three patients were administered dexmedetomidine only at 0.0065% concentration.

Following the administration, IOP was measured. In addition, immediately prior to the first administration, baseline IOP was measured.

### Experimental Results

The results of the experiment are summarized in Tables 3-7.

TABLE 3
(0.0055%) (Patient 1) (both eyes)

| | | Average | % of | Average | % of |
|---|---|---|---|---|---|
| | | IOP (right | Baseline | IOP (left | Baseline |
| Time | | eye) (mm | IOP (right | eye) (mm | IOP (left |
| Points | | of mercury) | eye) (%) | of mercury) | eye) (%) |
| Baseline | | 11 | 100 | 12.3 | 100 |
| 30 | min | 9.7 | 87.9 | 11.0 | 91.7 |
| 2 | hours | 6.7 | 60.9 | 7.7 | 64.2 |
| 3.5 | hours | 6.8 | 61.4 | 8.0 | 66.7 |

TABLE 4
(0.0065%) (Patient 1) (both eyes)

| | | Average | % of | Average | % of |
|---|---|---|---|---|---|
| | | IOP (right | Baseline | IOP (left | Baseline |
| Time | | eye) (mm | IOP (right | eye) (mm | IOP (left |
| Points | | of mercury) | eye) (%) | of mercury) | eye) (%) |
| Baseline | | 13 | 100 | 12 | 100 |
| 1 | hour | 7.3 | 56.2 | 7.7 | 64.2 |
| 2 | hours | 7.7 | 59.2 | 6.7 | 55.8 |
| 4 | hours | 8.0 | 61.5 | 7.7 | 64.2 |
| 5.5 | hours | 8.0 | 61.5 | 7.0 | 58.3 |
| 7 | hours | 9.3 | 71.5 | 9.0 | 75.0 |
| 8 | hours | 9.7 | 74.6 | 9.0 | 75.0 |
| 26 | hours | 10.0 | 76.9 | 10.0 | 83.3 |

As Table 4 demonstrates, topical application of 0.0065% dexmedetomidine to the left eye resulted in 44.2% reduction in IOP at the two hour time point versus the baseline. At 5.5 hours after the application, there was about 40% reduction in IOP versus the baseline. Even at 26 hours, there was still around 20% reduction in IOP which is a substantially lower reduction that that previously reported for conventional α-2 based glaucoma treatments.

TABLE 5
(0.0065%) (Patient 2) (left eye only)

| | Average | % of | Average | % of |
|---|---|---|---|---|
| | IOP (right | Baseline | IOP (left | Baseline |
| Time | eye) (mm | IOP (right | eye) (mm | IOP (left |
| Points | of mercury) | eye) (%) | of mercury) | eye) (%) |
| Baseline | 18 | 100 | 18 | 100 |
| 3.5 hours | 18 | 100 | 12 | 66.7 |

TABLE 6
(0.0065%) (Patient 3) (right eye only)

| | Average | % of | Average | % of |
|---|---|---|---|---|
| | IOP (right | Baseline | IOP (left | Baseline |
| Time | eye) (mm | IOP (right | eye) (mm | IOP (left |
| Points | of mercury) | eye) (%) | of mercury) | eye) (%) |
| Baseline | 13 | 100 | 13 | 100 |
| 3.5 hours | 10 | 76.9 | 13 | 100 |

TABLE 7
(0.0065%) (Patient 4) (right eye only)

| | Average | % of | Average | % of |
|---|---|---|---|---|
| | IOP (right | Baseline | IOP (left | Baseline |
| Time | eye) (mm | IOP (right | eye) (mm | IOP (left |
| Points | of mercury) | eye) (%) | of mercury) | eye) (%) |
| Baseline | 15 | 100 | 12 | 100 |
| 3.5 hours | 10 | 66.7 | 12 | 100 |

Patient 4 was suspected of having glaucoma and was on twice daily Alphagan-P® regimen at the time of the experiment. However, the patient was instructed not to apply Alphagan-P® to the right eye on the day of the experiment. The patient applied Alphagan-P® only to the left eye on the day of the experiment.

The experiment has demonstrated that topically applying dexmedetomidine at concentrations of 0.0055% and 0.0065% has resulted in a significant reduction of IOP in both eyes at various time points. Notably, dexmedetomidine at 0.0065% has resulted in reduction of IOP even 26 hours after the application.

### Example 2

### Effect of Topical Administration of Dexmedetomidine to the Eyes on Sedation, Dry Mouth and Eye Whitening

### Experimental Design

The purpose of this experiment was to evaluate side effects (such as sedation and dry mouth) of topical ocular delivery of dexmedetomidine. The experiment was designed as follows:

A patient was given one drop of dexmedetomidine at pH 7.4 at various concentrations in both eyes. Following the administration, eye whitening and the side effects of sedation and dry mouth were measured. The following scale of 1-4 was used to measure sedation:

1—trace (slight fatigue);

2—mild fatigue;

3—moderate fatigue (lethargic); and

4—severe fatigue (could not stay awake).

The dexmedetomidine was in the form of dexmedetomidine HCl formulation, prepared using BSS® diluent pH 7.1, final pH 6.7 (contains acetate buffer and sodium citrate dehydrate 0.17%).

### Experimental Results

The results of the experiment are summarized in Table 8.

TABLE 8

| Concentration | Sedation | Dry Mouth | |
|---|---|---|---|
| (w/v %) | (out of 4 max) | (out of 4 max) | Eye Whitening |
| 0.05 | N/A | N/A | N/A |
| 0.03 | 3 | 3 | moderate-strong |
| 0.02 | ≦1 | ≦1 | N/A |
| 0.007 | 0 | 0 | moderate |
| 0.0065 | 0 | 0 | moderate |
| 0.0055 | 0 | 0 | slight to moderate |

The experiment has demonstrated that the administered dexmedetomidine had a significant sedative effect at 0.03%; a very slight sedative effect and dry mouth effect at 0.01%, and no sedative or dry mouth or other side effect demonstrated at below 0.013%. Further, dexmedetomidine even at the lowest tested concentration of 0.0055% resulted in slight to moderate eye whitening, indicating that it did not induce hyperemia and retained its cosmetic α-2 induced vasoconstrictive properties, and at 0.0065% and 0.007% had more profound IOP lowering effect in normotensive eyes than previously demonstrated for this class.

### Example 3

Effect of pH and Diluent on Dexmedetomidine Formulations

### Experimental Design:

The purpose of this experiment was to evaluate the effect of different pH and diluent formulations of dexmedetomidine on the IOP. The experiment was designed as follows:

On separate occasions, a patient was administered two drops of one of Formulations A, B, or C, followed by one more drop of the same formulation in about 5 minutes after the first administration.

A baseline IOP was measured prior to administration, and was between 12 and 14 mm Hg. Then, the IOP was measured at one and a half hour and two hours following the administration of dexmedetomidine formulations.

### Formulation A

0.0065% dexmedetomidine HCl, using 0.01% Precedex® diluted with BSS®, caprilyic acid equimolar, pH of 7.4.

### Formulation B

0.0050% dexmedetomidine HCl using 0.01% Precedex® diluted with BSS®, pH of about 6.8-7.0

### Formulation C

0.007% dexmedetomidine HCl using powder and pH 7.1 BSS® as diluent (containing acetate buffer and sodium citrate dehydrate 0.17% to achieve a final pH of about 6.7.

### Experimental Results

Formulation A: the reduction of IOP was less than 10% one and a half hours following the administration, and about the same two hours following the administration.

Formulation B: the reduction of IOP was about 15% one and a half hours following the administration and was about 30% two hours following the administration.

Formulation C: the reduction of IOP was about 40% one and a half hours following the administration and was about 44.5% two hours following the administration.

### Example 4

### Effect of Topical Administration of Dexmedetomidine at 0.01% and 0.0065% on Cosmetic Appearance of the Eyes

### Experimental Design

The purpose of this experiment was to evaluate effect of dexmedetomidine at concentrations of 0.0065% and 0.01% at pH 6.7 (using BSS® pH 7.1 diluent) on cosmetic appearance (i.e., whiteness) of the treated eyes. The experiment was designed as follows:

A drop of dexmedetomidine at 0.0065% and 0.01% was topically applied to the eyes of an individual. Eye whiteness prior to and after the application was visually measured by the patient on a scale of 0 (white eye, no hyperemia) to 4 (significantly reddened eye, strong hyperemia).

### Experimental Results

Application of both 0.0065% and 0.01% dexmedetomidine caused a significant eye whitening effect.

Pre-application hyperemia was visually estimated by the patient examiner to be 1.25-1.5 out of 4.0 for both eyes.

About 15 minutes after 0.01% dexmedetomidine was applied, hyperemia was estimated to be ≦0.5 out of 4.0.

The patient's eyes started to whiten about 2 minutes after the application of 0.0065% dexmedetomidine; the maximum whiteness was at about 10 minutes (hyperemia was estimated to be 0-0.5 out of 4.0) with gradual and slow decline thereafter; and the total duration of the whitening effect was about 4-5 hours.

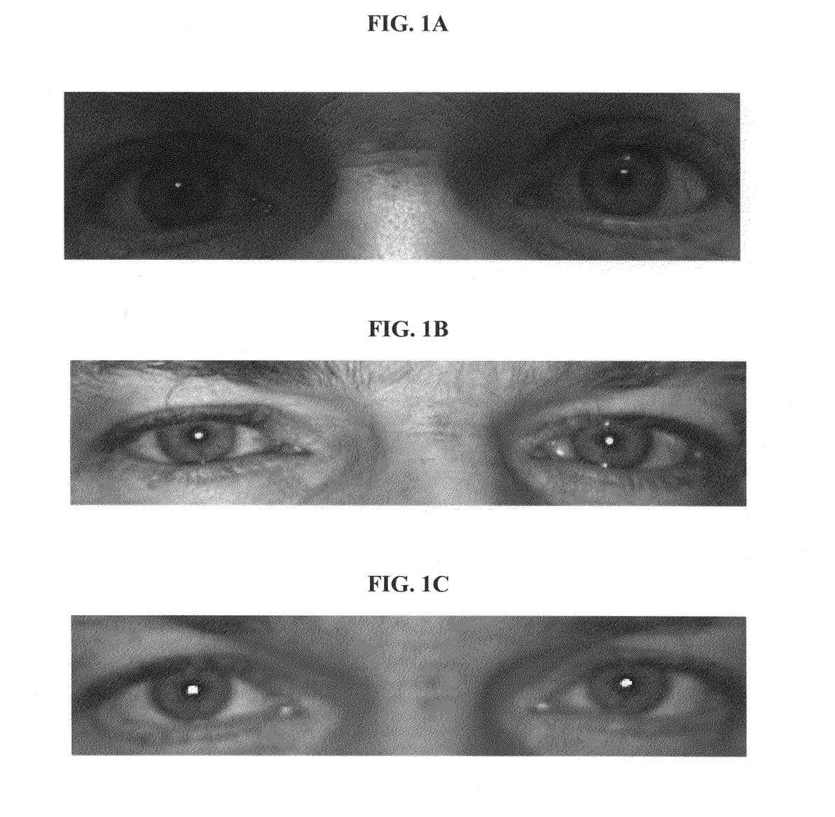

FIG. 1A is a color photograph of the treated eyes taken about 15 minutes after dexmedetomidine at 0.01% was applied. Pre-application hyperemia was visually estimated by the patient examiner to be 1.25-1.5 out of 4.0; post-application hyperemia was estimated to be ≦0.5 out of 4.0.

FIG. 1B is a color photograph of the eyes taken pre-application of 1 drop of dexmedetomidine at 0.0065% to both eyes. Pre-application hyperemia was estimated to be 1.5 out of 4.0 for both eyes.

FIG. 1C is a color photograph of the same eyes as in FIG. 1B taken about 10 minutes after dexmedetomidine at 0.0065% was applied. Post-application hyperemia was estimated to be 0-0.5 out of 4.0. The eyes started to whiten about 2 minutes after the application; the maximum whiteness was at about 10 minutes with gradual and slow decline thereafter; and the total duration of the whitening effect was about 4-5 hours.

The experiment has demonstrated that dexmedetomidine at 0.0065% provides excellent hyperemia reduction and effects even more cosmetic improvement via eye whitening.

### Example 5

### Effect of Dexmedetomidine at 0.0133% Vs. Adding Dexmedetomidine at 0.0133% to Xalatan® (Latanoprost) on the Reduction of IOP

### Experimental Design

The purpose of this experiment was to compare the effect on reducing the IOP of a combination of Xalatan® and dexmedetomidine at 0.0133% versus dexmedetomidine at 0.0133% alone.

The experiment was designed as follows:

At 0 hr, a baseline IOP in both eyes of a patient was measured prior to administration, and was about 18 mm Hg. Then, a drop of Xalatan® was applied to the right eye of a patient and a drop of dexmedetomidine at 0.0133% was applied to the left eye of the patient. 3 hours after administration, a drop of dexmedetomidine at 0.0133% was applied to the right eye of the patient. Measurements of the IOP in both eyes were taken at 0, 3, 5, 8, and 16 hours following the administration. Redness of both eyes was also visually estimated.

### Experimental Results

The results of the experiment on the reduction in the IOP are summarized in Table 9.

TABLE 9

| | | | | % of | % of |
|---|---|---|---|---|---|
| | | IOP left | IOP right | base- | base- |
| Time after | | eye (mm of | eye (mm of | line IOP | line IOP |
| administration | | mercury) | mercury) | (left eye) | (right eye) |
| 0 | hr | 18 (initial IOP) | 18 (initial IOP) | 100% | 100% |
| 3 | hrs | 11 (dex alone) | 14 (Xalatan ® | 61.1% | 77.8% |
| | | | alone) | | |
| 5 | hrs | 10 (dex alone) | 8.5 (Xalatan ® | 55.6% | 47.2% |
| | | | plus dex) | | |
| 8 | hrs | 10 (dex alone) | 7.5 (Xalatan ® | 55.6% | 41.7% |
| | | | plus dex) | | |
| 16 | hrs | 16 (dex alone) | 16 (Xalatan ® | 88.9% | 88.9% |
| | | | plus dex) | | |

The experiment has demonstrated that: 1) dexmedetomidine at 0.0133% alone is highly effective in reducing the IOP and 2) dexmedetomidine at 0.0133% can be combined with Xalatan® to provide a reduction in the IOP.

With respect to redness, dexmedetomidine at 0.0133% resulted in a significant reduction of redness at 3 hr and 5 hr time points.

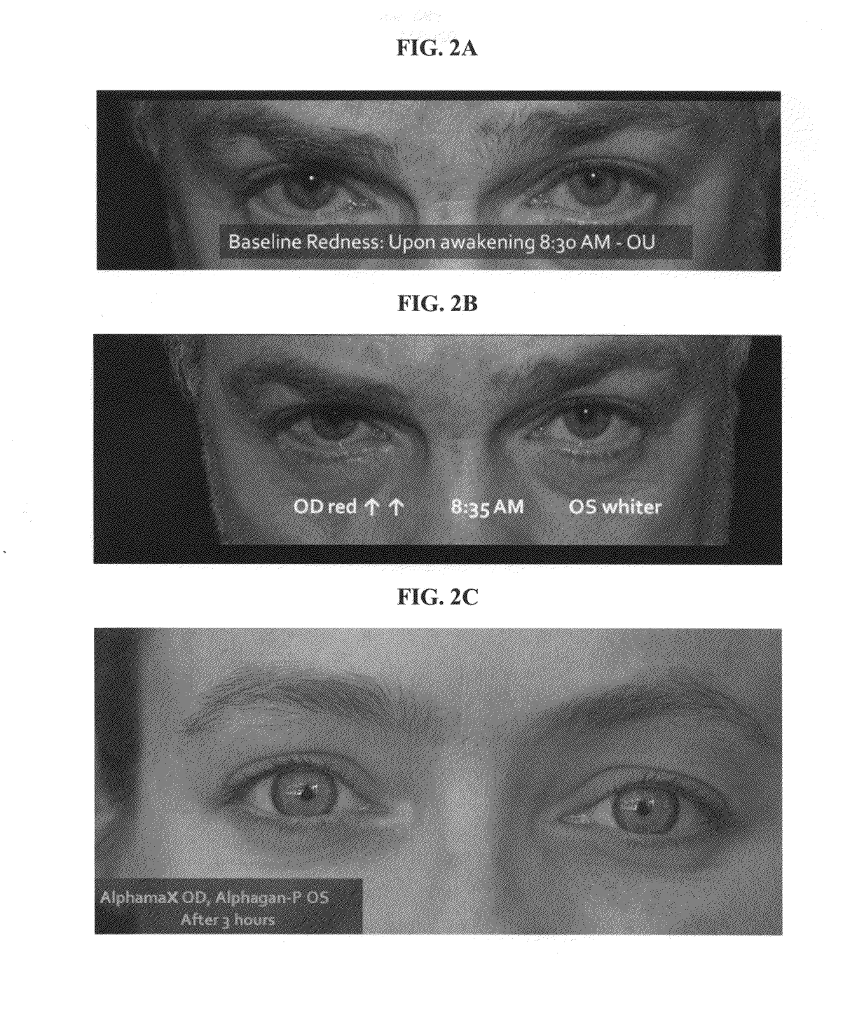

FIG. 2A is a photograph of a patient's eyes immediately prior to application of 0.0133% dexmedetomidine (OS; left eye) and Xalatan® (latanoprost) (OD; right eye). One can see that both eyes have significant baseline redness.

FIG. 2B is a photograph of the same eyes as in FIG. 3A 5 minutes after application of 0.0133% dexmedetomidine (OS; left eye) and Xalatan® (latanoprost) (OD; right eye). One can see that the left eye (OS) is significantly whiter than the right eye (OD), indicating dexmedetomidine's effectiveness in reducing eye redness.

FIG. 2C is a photograph of the same patient's eyes following 3 hour application of 0.0133% dexmedetomidine (OS; left eye) and Xalatan® (latanoprost) (OD; right eye). Both eyes appear significantly whiter at this point compared to their baseline redness.

What is claimed is:

1. A pharmaceutical composition effective for the treatment of glaucoma in a patient in need thereof comprising a selective α-2 adrenergic receptor agonist having a binding affinity of 1000 fold or greater for α-2 over α-1 adrenergic receptors at a concentration from between about 0.001% to about 0.025% weight by volume, wherein said pharmaceutical composition has a final pH of 6.5 or greater.

2. The pharmaceutical composition of claim 1, wherein said final pH is between about 6.5 and about 7.0.

3. The pharmaceutical composition of claim 1, wherein said selective α-2 adrenergic receptor agonist is present at a concentration from about 0.0025% to about 0.0065% weight by volume.

4. The pharmaceutical composition of claim 1, wherein said selective α-2 adrenergic receptor agonist has a binding affinity of 1500 fold or greater for α-2 over α-1 adrenergic receptors.

5. The pharmaceutical composition of claim 1, wherein said selective α-2 adrenergic receptor agonist is dexmedetomidine.

6. A pharmaceutical composition effective for the treatment of glaucoma in a patient in need thereof comprising a selective α-2 adrenergic receptor agonist having a binding affinity of 1000 fold or greater for α-2 over α-1 adrenergic receptors at a concentration from between about 0.001% to about 0.025% weight by volume, wherein said selective α-2 adrenergic receptor agonist has an octanol-water partition coefficient Log D of between about 2.40 and about 3.00.

7. The pharmaceutical composition of claim 6, wherein said octanol-water partition coefficient is between about 2.50 and 2.85.

8. The pharmaceutical composition of claim 6, wherein said selective α-2 adrenergic receptor agonist is dexmedetomidine.

9. The pharmaceutical composition of claim 6, further comprising:
a. carboxymethyl cellulose at a concentration of between about 0.1% and about 0.5% weight by volume;
b. mannitol at a concentration of between about 1% and about 4% weight by volume and
c. 2-hydroxypropyl-beta cyclodextrin at a concentration of between about 0.5% and about 5% weight by volume;
wherein said pharmaceutical composition has a pH of about 7.0 or greater.

10. An aqueous pharmaceutical composition effective for the treatment of glaucoma of a patient in need thereof comprising:
a. dexmedetomidine at a concentration from between about 0.0025% to about 0.025% weight by volume,
b. citrate at a concentration of between about 0.10% and about 0.5% or EDTA at a concentration of between about 0.005% and about 0.02%
c. carboxymethyl cellulose at a concentration of between about 0.1% and about 0.3% weight by volume;
d. acetate buffer at a concentration of between about 1 mM and about 100 mM; and
e. sodium chloride at a concentration of between about 0.3% and about 0.75% and/or potassium chloride at a concentration of between about 0.05% and about 0.15% and/or calcium chloride at a concentration of between about 0.02% and about 0.05%,
wherein said aqueous pharmaceutical composition has a final pH of about 6.5 or greater.

11. A method of treating glaucoma comprising administering to a patient in need thereof the pharmaceutical composition of claim 1.

12. The method of claim 11, wherein said method provides eye whitening and reduction in intraocular pressure compared to a baseline of said patient.

13. A method of improving corneal permeation of dexmedetomidine in a patient having glaucoma comprising administering to said patient the pharmaceutical composition of claim 1.
